(19) United States
(12) Patent Application Publication
(10) Pub. No.: US 2018/0296615 A1
(43) Pub. Date: Oct. 18, 2018

(54) PHARMACEUTICAL COMPOSITION OR FUNCTIONAL HEALTH FOOD FOR PREVENTING AND TREATING METABOLIC DISEASES, CONTAINING WATER EXTRACT OF PLEUROTUS ERYNGII VAR. FERULAE (PF.) AS ACTIVE INGREDIENT

(71) Applicants: KYONGSANGBUK-DO, Daegu (KR); POSTECH ACADEMY-INDUSTRY FOUNDATION, Pohang-si, Gyeongsangbuk-do (KR)

(72) Inventors: Sung Ho RYU, Pohang-si (KR); Jaewang GHIM, Pohang-si (KR); Jong In KIM, Pohang-si (KR); Jaeyoon KIM, Pohang-si (KR); Yongmi KIM, Pohang-si (KR); Youngwoo CHOI, Pohang-si (KR); Kyung-Jin JO, Pohang-si (KR); Wook Dong KIM, Daegu (KR); Sang Kap SHIM, Andong-si (KR); Woo Jae CHEON, Pohang-si (KR); Jong Hyeok NAM, Andong-si (KR)

(73) Assignees: KYONGSANGBUK-DO, Daegu (KR); POSTECH ACADEMY-INDUSTRY FOUNDATION, Pohang-si, Gyeongsangbuk-do (KR)

(21) Appl. No.: 15/576,768

(22) PCT Filed: May 9, 2016
(86) PCT No.: PCT/KR2016/004806

§ 371 Date: Nov. 24, 2017

(30) Foreign Application Priority Data
May 27, 2015 (KR) 10-2015-0073680

(51) Int. Cl. A61K 36/07; A61P 3/04; A61P 3/10; A61K 9/16; A61K 9/20; A61K 9/48; A61K 9/08; A61K 47/26; A61K 9/00; A61K 47/02; A23L 31/00; A23L 33/10

(52) U.S. Cl. CPC A61K 36/07; A23V 2002/00; A61P 3/10; A61K 9/1623; A61K 9/1611; A61K 9/2059; A61K 9/2018; A61K 9/2013; A61K 9/4858; A61K 9/4866; A61K 9/08; A61K 47/26; A61K 9/0019; A61K 47/02; A61K 9/0056; A23L 31/00; A23L 33/10; A61K 2236/331; A61P 3/04

(57) ABSTRACT

The present invention relates to a composition for preventing and treating metabolic disorders, containing a water extract of Pleurotus eryngii var. ferulea (Pf) as an active ingredient, wherein the water extract of Pleurotus eryngii var. ferulea (Pf) has a remarkable effect of being able to be used as a pharmaceutical composition or functional health food for preventing or treating obesity or diabetes by confirming the functionality whereby fat accumulation and blood glucose increase generated when taking a high fat diet can be inhibited.

### FIELD OF INVENTION

The present invention pertains to a composition or a functional health food for preventing and treating metabolic diseases, containing a water extract of Pleurotus eryngii var. ferulea (PF.) as an active ingredient and, more specifically to a pharmaceutical composition or a functional health food for preventing and treating metabolic diseases such as obesity and diabetes, containing a water extract of Pleurotus eryngii var. ferulea (PF.) inhibiting fat accumulation and increase in blood sugar caused by intake of high-fat meal.

### BACKGROUND OF THE INVENTION

People can enjoy foods fluently thanks to improvement of standard of living according to the recent economic development, but change of diet into meat-centered diet caused intake of excessive calories. This change of diet of modern people shows the trend of increase in obese population due to less calories consumption because of lack of exercise. Obesity is reported to cause not only simple problems in appearance, but also various diseases of adult diseases such as high blood pressure, diabetes, hyperlipidemia and coronary artery and breast cancer, uterine cancer, colorectal cancer and the like by being continued, and is considered as one of fatal diseases [J. Biol. Chem., 273, 32487˜32490 (1998); Nature, 404, 652˜660 (2000)].

Metabolic diseases include diseases caused by imbalance of sugar, lipid, protein, vitamin, mineral, moisture and the like. Metabolic diseases related to bilayer lipid thereof mean diseases caused by excessive fat accumulation in a living body, and obesity and diabetes are one of said diseases.

Obesity is caused by imbalance between intake and consumption of energy, and the surplus energy is converted into a form of fat cell and stored in the body. Specifically, about 20 billion fat cells exist in a human body and accumulate or discharge energy in a living body of mammals, and the fat cells stores energy remaining after consumption in a form of triglycerides and decompose the same into free fatty acid and glucose again when energy is depleted. When excessive energy is accumulated due to imbalance of the process of storage and decomposition, the size and number of fat cells are increased and causes obesity.

Diabetes is divided into insulin dependent diabetes (type 1 diabetes), insulin-independent diabetes (type 2 diabetes) and malnutrition-related diabetes mellitus (MRDM), and type 2 diabetes occupying 90% or more of Korean diabetes patients is a metabolic disease characterized by high blood sugar and is reported to be caused by decrease in insulin secretion of pancreas beta cells and increase in insulin resistance of peripheral tissues due to genetic, metabolic and environmental factors.

Diabetes is closely related to obesity and prevalence mechanism, and thus if the body fat is increased due to obesity, the insulin sensitivity is decreased, and there is a close relation between obesity and insulin resistance in a patient having type 2 diabetes, and thus more severe the obesity is, the higher the insulin resistance is.

Current treating agents for treating obesity is broadly divided into a medicine influencing appetite by applying to the central nervous system and a medicine inhibiting absorption by applying to the gastrointestinal tract. As the medicine applying to the central nervous system, medicines such as fenfluramine and dexfenfluramine inhibiting serotonin (5HT) nervous system, medicines such as ephedrine and caffeine applying to noradrenalin nervous system and recently medicines such as sibutramine inhibiting obesity by simultaneously applying to serotonin and noradrenaline nervous system are on the market. Furthermore, one of representative medicines inhibiting obesity by applying to the gastrointestinal tract is orlistat which is recently proved as an obesity treating agent by reducing fat absorption by inhibiting lipase generated in the pancreas.

However, fenfluramine and the like of obesity treating agents which have been used before cause side effects such as primary pulmonary hypertension or valvular lesion and thus was recently prohibited, sibutramine has a side effect of increasing blood pressure and orlistat was known as its side effect of disorders of the digestive organ. In addition, other chemical synthetic medicines cause problems such as blood pressure reduction and lactacidema and thus cannot be used for patients having heart failure, renal disease and the like.

Therefore, a method for preventing or treating obesity and relevant diabetes with low side effects is required nowadays and recently, researches are actively carried out to find solutions from natural ingredients.

There are about 10,000 types of mushrooms reported over the world, and they have high edibility and medicinal use value, and thus many researches are carried out in the developed countries such as Europe, USA, Japan and the like to obtain mushrooms as an effective microorganism resource. Especially, there are many study results that bioactive materials produced by mushrooms have less side effect to be safe with respect to toxicity and have various functions such as function control of immune systems in a human body, an anti-cancer effect and metabolism control.

Pleurotus eryngii var. ferulea (PF.) naturally growing in China and Central Asia is known as a medicinal plant which opens up the blocked passages, reduces coughs and inflammation and treats gastrointestinal diseases, and is a high functional mushroom having the effects of discharging toxin in a human body, stopping coughs, reducing inflammation and treating obstetric and gynecologic diseases introduced in oriental medicinal books.

The scientific name is Pleurotus eryngii var. ferulea (Pf: P. ferulea) which is a variant of King Oyster Mushroom. The English name is Ferula Oyster Mushroom. Pleurotus eryngii var. ferulea (PF.) is called Baek Ryung Cheuk Yi in China. Pleurotus eryngii var. ferulea (PF.) naturally grows on P. ferulea tree in Xinjiang which is a dry area in China, the growth temperature is middle temperature of 8-20° C., and thus the proper cultivation season in Korea is spring and fall.

The prior art of the present invention is an anti-inflammation composition for external skin use containing a Pleurotus ferulea fruit body extract or a Pleurotus ferulea mycelium extract or a Pleurotus ferulea mycelium culture fluid published as Korean patent publication No. 10-1402193, but the patent relates to an anti-inflammation composition comprising a Pleurotus ferulea fruit body extract or a Pleurotus ferulea mycelium extract as an active ingredient.

Meanwhile, the inventers of the present invention published composition for preventing and treating hyperlipidemia, containing a water extract of Pleurotus eryngii var. ferulea (PF.) as an active ingredient in Korea patent application No. 10-2012-0143701 (divisional application No. 10-2015-0055605), and during the research on the water extract of Pleurotus eryngii var. ferulea (PF.), another effect of the extract inhibiting fat accumulation and blood sugar increase caused by intake of high fat meal was discovered, and the present invention was completed based on the effect which has not been published yet.

### DETAILED DESCRIPTION OF THE INVENTION

### Technical Subject

Accordingly, the present invention has the purpose of providing a pharmaceutical composition for preventing and treating metabolic diseases such as obesity or diabetes, containing a water extract of Pleurotus eryngii var. ferulea (PF.) as an active ingredient.

Another purpose of the present invention is to provide a health functional food for the prevention and alleviation of metabolic disorders such as obesity or diabetes, containing a water extract of Pleurotus eryngii var. ferulea (PF.) as an active ingredient.

### Means for Solving Problems

The present invention is achieved by obtaining a water extract of Pleurotus eryngii var. ferulea (PF.), verifying and evaluating the anti-obesity or anti-diabetic functionality of the same as a test material.

### Effects of the Invention

A water extract of Pleurotus eryngii var. ferulea (PF.) of the present invention is functional to inhibit fat accumulation and glucose increase which occur when having high fat meal and thus can provide a pharmaceutical composition for preventing and treating the metabolic diseases such as obesity or diabetes, containing the same as an active ingredient and can provide an effect of being used as a functional health food for prevention and alleviation of metabolic diseases such as obesity or diabetes.

### BRIEF DESCRIPTION OF DRAWINGS

FIG. 1 is a graph showing the weight change of the experimental animals depending on a water extract of Pleurotus eryngii var. ferulea (PF.).

FIG. 2 is a graph showing the total adipose tissue weight changes in experimental animals depending on a water extract of Pleurotus eryngii var. ferulea (PF.).

FIG. 3 is a diagram showing the morphological changes in the adipose tissues of experimental animals depending on a water extract of Pleurotus eryngii var. ferulea (PF.).

FIG. 4 is a diagram showing the change in fat accumulation in the liver of experimental animals depending on a water extract of Pleurotus eryngii var. ferulea (PF.).

FIG. 5 is a graph showing the blood glucose change in the experimental animal depending on a water extract of Pleurotus eryngii var. ferulea (PF.).

### DETAILED DESCRIPTION OF THE EMBODIMENTS

A water extract of Pleurotus eryngii var. ferulea (PF.) of the present invention is manufactured by the steps of: preparing a Pleurotus eryngii var. ferulea (PF.) powder; mixing the Pleurotus eryngii var. ferulea (PF.) powder with water; and extracting the same mixture at a temperature of 20 to 60° C. for 12 to 36 hours.

The water extract of Pleurotus eryngii var. ferulea (PF.) may be one prepared according to the conventional method for preparing plant extracts. Most preferably, the Pleurotus eryngii var. ferulea (PF.) is ground after cold and hot dry of 15° C., the residue is removed, 0.1 to 20 g of the ground product per water 100 mL were added, most preferably 1 to 5 g of ground product per 100 mL of extraction solvent was added, and the extraction is performed. 40° C. or more hot air drying is not preferred. In addition, if the content of ground Pleurotus eryngii var. ferulea (PF.) is too little than the extraction solvent, the cholesterol absorption of Pleurotus eryngii var. ferulea (PF.) is not sufficiently made which is not preferred, and if the content of ground Pleurotus eryngii var. ferulea (PF.) is too greater than the amount of extraction solvent, the improvement of effect is not increased while the production cost is increased which is not preferred in terms of productivity.

The extraction condition of Pleurotus eryngii var. ferulea (PF.) is preferably mixing Pleurotus eryngii var. ferulea (PF.) with the extraction solvent of water and extracting the same at 20-60° C. for 12 to 36 hours, most preferably extracting at a temperature of 30 to 40° C. for 20 to 24 hours. When extracting at the low temperature conditions of 20° C. or less, a long time is required for the extraction of the active extraction ingredient, and if extracting at a high temperature condition of 60° C. or more, the activity is reduced which is not preferable. In particular, a water extraction product hot extracted with water at 100° C. for 15 minutes has no activity and thus could not be used. If the extraction time is 12 hours or shorter, concentration of the extracted active ingredient is low, and if the extraction time is 36 hours or more, increase in concentration of the extracted active ingredients in accordance with the increase in extraction time is little, and thus the condition is not preferable in terms of productivity. The water extract of Pleurotus eryngii var. ferulea (PF.) extracted by the same method was preferred to be used by filtering with filter fabric or the like, centrifuging the filtrate, removing the precipitate, concentrating under reduced pressure or concentrating and then freeze-drying the same.

On the other hand, the water extract of Pleurotus eryngii var. ferulea (PF.) prepared according to the described embodiment may be contained as an active ingredient in a pharmaceutical composition for preventing and treating metabolic diseases such as obesity or diabetes. The amount of the water extract of Pleurotus eryngii var. ferulea (PF.) which is an active ingredient of the pharmaceutical composition for the prevention and treatment of metabolic disorders such as obesity or diabetes is preferably 0.01 to 30 wt %. If the content of the water extract of Pleurotus eryngii var. ferulea (PF.) which is an active ingredient is less than 0.01 wt %, the effect of inhibiting fat accumulation and blood sugar increase caused by intake of high fat meal is insignificant, and if the content exceeds 30 wt %, increase in the inhibitory effect in accordance with the increase in content is insignificant and thus the condition is not economical. Preferably, the content of the water extract of Pleurotus eryngii var. ferulea (PF.) in the composition is 0.001 to 50 wt %, the most preferably 0.1 to 30 wt %.

A pharmaceutical composition comprising such the water extract of Pleurotus eryngii var. ferulea (PF.) as an active ingredient may further comprises suitable carriers, excipients, and diluents commonly used in the manufacture. As carriers, excipients, and diluents that may be included in pharmaceuticals containing the water extract of Pleurotus eryngii var. ferulea (PF) as an active ingredient of the present invention are lactose, dextrose, sucrose, sorbitol, mannitol, xylitol, erythritol, maltitol, starch, acacia rubber, alginate, gelatin, calcium phosphate, calcium silicate, cellulose, methyl cellulose, microcrystalline cellulose, polyvinylpyrrolidone, water, methyl hydroxybenzoate, propyl hydroxybenzoate, talc, magnesium stearate and mineral oil.

A pharmaceutical composition comprising a water extract of Pleurotus eryngii var. ferulea (PF.) of the present invention as an active ingredient may be used in the oral formulation of powders, tablets, hard and soft capsules, liquid preparation in a usual manner, respectively. The oral formulation is intended to include a solid preparation and a liquid preparation for oral administration, and a solid preparation for oral administration may include tablets, pills, powders, granules, capsules and the like. Such solid formulations can be prepared by mixing the extract with at least one excipient, for example, starch, calcium carbonate, sucrose or lactose, gelatin and the like. Further, a lubricant such as magnesium stearate and talc other than a simple excipient can be used. Liquid formulations for oral administrations are suspensions, solutions, emulsions, syrup and the like, and various excipients except for water and liquid paraffin which are simple diluents commonly used, for example, wetting agents, sweeteners, aromatics and preservatives and the like can be included.

The preferred dosage of the pharmaceutical composition containing a water extract of Pleurotus eryngii var. ferulea (PF.) according to the present invention depends on the condition, body weight, level of disease, drug form, route of administration, and duration but may be suitably selected by a person skilled in the art. Preferably, with respect to the amount of medicines containing the water extract of Pleurotus eryngii var. ferulea (PF.) as an active ingredient of the present invention, 0.0001 to 100 mg/kg is administered to an adult (60 kg of body weight) a day based on the amount of the water extract of Pleurotus eryngii var. ferulea (PF.), more effectively 0.01-10 mg/kg is administered. The number of administration may be once a day and may be divided into several times. The dose and number of administration are in any aspects not intended to limit the scope of the invention.

According to another embodiment of the present invention, provided is a health functional food for the treatment and alleviation of metabolic disorders such as obesity or diabetes, containing a water extract of Pleurotus eryngii var. ferulea (PF.) as an active ingredient. “The health functional food” in this description refers to a natural product or a processed product which contains one or more nutrients, and preferably means that the state-to-eat directly after a certain processing step.

A functional health food to which the water extract of Pleurotus eryngii var. ferulea (PF.) of the present invention can be added is for example, a variety of foods, drinks, gum, tea, vitamin complex and the like. In addition, the food of the present invention includes, but is not limited to special nutrition food (e.g. prepared oils, baby food and the like), processed meat products, fish products, tofu, jellied foods, noodles (e.g. ramen, noodles and the like), dietary supplements, seasoning products (e.g. soy sauce, soybean paste, red pepper paste, mixed paste and the like), sauces, confectionery (e.g. snacks), dairy products (e.g. fermented milk, cheese and the like), and other processed foods, kimchi, pickled food (various kimchi, pickles and the like), beverages (e.g. fruit and vegetable beverages, soy milk, fermented beverages and the like), natural flavoring (e.g. ramen soup and the like). The food, drink or food additives may be manufactured by a conventional manufacturing method.

In this specification, the functional food is foods provided with an added value to apply and express the function of the food for a particular purpose by using the physical, biochemical, biotechnological techniques on foods or processed foods designed to fully perform the body control function related to biological defense rhythm adjustment, disease prevention and recovery of food compositions in vivo. The functional foods may include sitologically-acceptable food supplement additives and may further comprise suitable carriers, excipients and diluents commonly used in the manufacture of functional foods.

In this specification, the beverage means a generic name of what is drunk to reduce thirst or enjoy the taste and is intended to include functional beverages. The beverage does not have any limit on other ingredients other than containing a water extract of Pleurotus eryngii var. ferulea (PF.) as an active ingredient as a necessary ingredient in the indicated ratio and may contain various flavors or natural carbohydrates as additional ingredients like conventional beverages. The examples of the natural carbohydrate are a monosaccharide such as glucose, fructose and the like, disaccharide such as maltose, sucrose and the like and polysaccharides such as a conventional sugar like dextrin and cyclodextrin, and sugar alcohol such as xylitol, sorbitol, erythritol and the like. As flavoring agents other than those described above, natural flavors (thaumatin, stevia extract (e.g. rebaudioside A, glycyrrhizin and the like)) and synthetic flavors (saccharin, aspartame and the like) can be advantageously used. The content of the natural carbohydrate may be a generally 1 to 20 g per 100 mL of the composition of the present invention, preferably 5 to 12 g. The composition of the present invention may further contain a pulp used in preparing natural fruit juice, fruit juice drink and vegetable drink.

A functional health food of the present invention in addition to the above may contain various nutrients, vitamins, minerals (electrolytes), flavors such as synthetic flavors and natural flavors, coloring agents and flavor enhancers (cheese, chocolate and the like), pectic acid and salts thereof, alginic acid and salts thereof, organic acid, protective colloidal thickeners, pH adjusting agents, stabilizers, preservatives, glycerin, water, acid agents used in soft drinks and the like. Such ingredients can be used independently or in a combination. The ratio of the additive may be not very critical and may be selected from 0 to 20 parts by weight per 100 parts by weight of the water extract of Pleurotus eryngii var. ferulea (PF.) of the present invention.

The functional beverage in the present invention is beverages provided with an added value to apply and express the function of beverage for a particular purpose by using the physical, biochemical, bioengineering techniques on the beverage or processed beverage designed to fully perform the body control function related to biological defense rhythm adjustment, disease prevention and recovery of beverage compositions in vivo.

The functional beverages do not have any limit on other ingredients other than containing a water extract of Pleurotus eryngii var. ferulea (PF.) as an active ingredient as a necessary ingredient in the indicated ratio and may contain various flavors or natural carbohydrates as additional ingredients like conventional beverages. The examples of the natural carbohydrate are a monosaccharide such as glucose, fructose and the like, disaccharide such as maltose, sucrose and the like and polysaccharides such as a conventional sugar like dextrin and cyclodextrin, and sugar alcohol such as xylitol, sorbitol, erythritol and the like. As flavoring agents other than those described above, natural flavors (thaumatin, stevia extract (e.g. rebaudioside A, glycyrrhizin and the like)) and synthetic flavors (saccharin, aspartame and the like) can be advantageously used. The content of the natural carbohydrate may be a generally 1 to 20 g per 100 mL of the composition of the present invention, preferably 5 to 12 g.

In addition, with respect to a health functional food for the purpose of the treatment or alleviation of metabolic disorders such as obesity or diabetes, the extract may be contained by 0.01 to 15 wt % of the total food weight, and a beverage compositions may contain 0.02-5 g, preferably 0.3-1 g of the extract based on 100 mL.

Hereinafter, the detailed description of the present will be disclosed with the embodiments. The following embodiments are only intended to provide examples, and the present invention is not limited to the embodiments.

### Example 1. Preparation of a Water Extract of Pleurotus eryngii Var. Ferulea (PF.)

The dried Pleurotus eryngii var. Ferulea (Pf) is obtained in the market, coarse-pulverized, put into a gauze bag, mixed with water as an extraction solvent by 50 mL per 1 g of the powder, and then extracted at 37° C. for 24 hours in a shaking incubator. A supernatant collected by centrifuging a supernatant of a shaking incubator with 2500 rpm for 10 minutes and then filtering the same was used as the following test material.

### Example 2. Anti-Obesity and Anti-Diabetic Efficacy Test of a Water Extract of Pleurotus eryngii Var. Ferulea (PF.)

In order to verify the anti-obesity and anti-diabetic efficacy of a water extract of Pleurotus eryngii var. ferulea (PF.), body weight gain, adipose tissue weight, morphological changes in fat and liver tissue and blood glucose content were measured by using experimental animals.

### Example 2-1: Experimental Animals and Diet

With respect to the experimental animals, C57BL/6-line 8-week-old male mice were adapted to solid feed in Postech experiment animal center for one week, mice having average weight of 25 g were divided into three groups by the randomized block design, and six mice for each group were raised for 8 weeks. The experimental group were used in the experiment by being divided into a normal diet group (NCD), a high-fat diet group (HFD) and a group fed with high fat diet and the fat and of a water extract of Pleurotus eryngii var. ferulea (PF.) (HFD+PEF). The normal diet group was provided with meals in which 10% of total calories are fat, the high fat diet group was provided with meals in which 60% of total calories are fat, and the group fed with high fat diet with the water extract of Pleurotus eryngii var. ferulea (PF.) was provided with high-fat diet meals containing 8 wt % of the water extract of Pleurotus eryngii var. ferulea (PF.). The groups were free to have water and feed during breeding period. The animal breeding room temperature was maintained to 22±1° C. and the lighting was controlled in 12-hour cycle (08:00-20:00), and all animal experiments were performed complying with ethical standards of animal experimentation with the approval of Pohang University of Science and Technology Institutional Animal Care and Use Committee.

### Example 2-2: Weight Measurement

Food intake and body weight of the experimental animals were measured once a week. Weight gain of each experimental group was measured at a regular time every week intervals during the experiment, and food efficiency ratio (FER) was calculated by dividing the weight increase during the experimental period by the feed intake during the experimental period as in Mathematical formula 1 by setting the experimental period from the experimental diet feeding day to sacrifice day.

Mathematical Formula 1

Food efficiency ratio (%)=total weight increase (g)/total diet intake (g) [Formula 1]

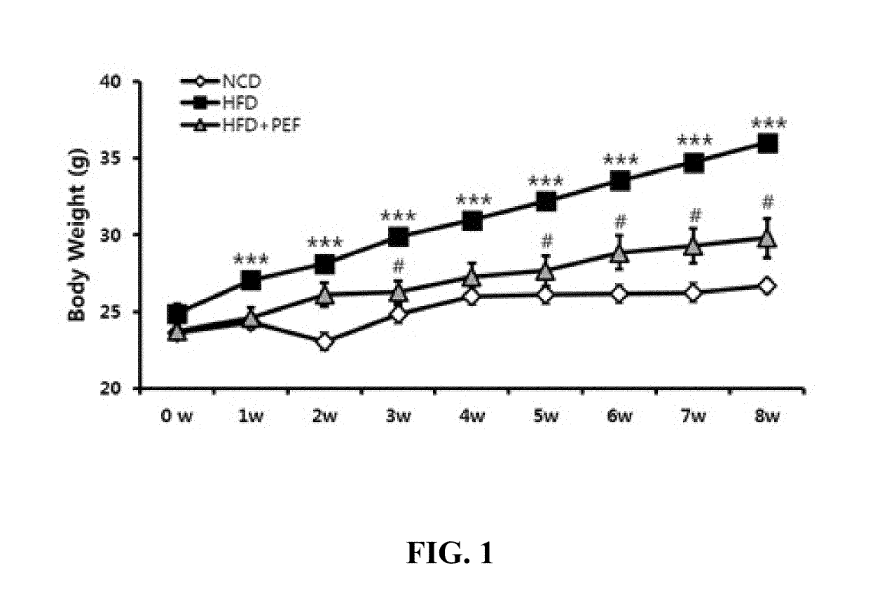

As the result of checking the weight for 8 weeks as in FIG. 1, the group which consumed high-fat diet (HFD) has significant increase in weight compared to the group which consumed normal diet (NCD), whereas remarkable reduction of weight gain.

In addition, the food efficiency ratio of the high fat diet group (HFD) was about 4.3 times greater than that of the normal diet group (NCD), but the food efficiency ratio of the group which had high fat diet and the water extract of Pleurotus eryngii var. ferulea (PF.) (HFD+PEF) was reduced by about 1.8 times.

TABLE 1
Comparison of food efficiency ratio depending on water
extract of Pleurotus eryngii var. ferulae (PF.)

| | Total body | Total | Food | |
|---|---|---|---|---|
| Experimental | weight | dietary | efficiency | |
| group | gain (g) | intake (g) | ratio (%) | p-value |
| Normal diet | 3.1 ± 0.3 | 23.45 ± 2.1 | 13.2 ± 0.3 | 0.048 |
| group (NCD) | | | | |
| High fat diet | 11.14 ± 0.8 | 19.17 ± 1.3 | 58.1 ± 13.3 | 0.000 |
| group (HFD) | | | | |
| High fat diet + | 6.1 ± 0.7 | 19.46 ± 1.5 | 31.4 ± 6.5 | 0.048 |
| water extract of | | | | |
| Pleurotus eryngii | | | | |
| var. ferulae (PF.) | | | | |

Therefore, it is confirmed that the weight gain caused by high fat diet can by significantly controlled when having the water extract of Pleurotus eryngii var. ferulea (PF.).

### Example 2-3: Verification of Anti-Obesity Effect

In order to check the anti-obesity effect of the water extract of Pleurotus eryngii var. ferulea (PF.), changes in the weight of the adipose tissue the size of fat cells of each group were observed with a microscope.

First, the entire adipose tissue of the experimental animals was extracted, and the weight was measured to determine the inhibitory effect of the water extract of Pleurotus eryngii var. ferulea (PF.) on the growth of adipose tissue.

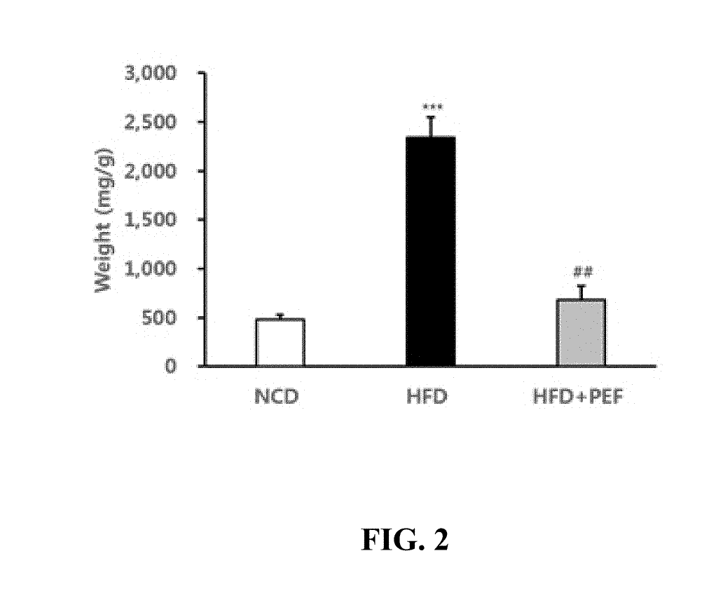

As shown in FIG. 2, the total weight of the adipose tissue of the normal diet group (NCD) was 0.47 g, and the total weight of the adipose tissue of the high fat diet group (HFD) was 2.3 g which is a high level, but the total weight of the adipose tissue of the group which had high fat diet and the water extract of Pleurotus eryngii var. ferulea (PF.) (HFD+PEF) was 0.67 g which means the total adipose tissue was reduced.

Next, the adipose tissue (WAT) and the liver tissue extracted from the experimental animals 8 weeks after the start of experiment were fixed in 4% paraformaldehyde solution for the morphologic observations of fat and liver tissue. Each tissue completely fixed was washed with water and then dehydrated with ethanol in the order of concentration increase, went through penetration and embedded on paraffin to make 4 μm sections of the tissue. A hematotaxyin and eosin staining was carried out, and the tissues were observed with an optical microscope.

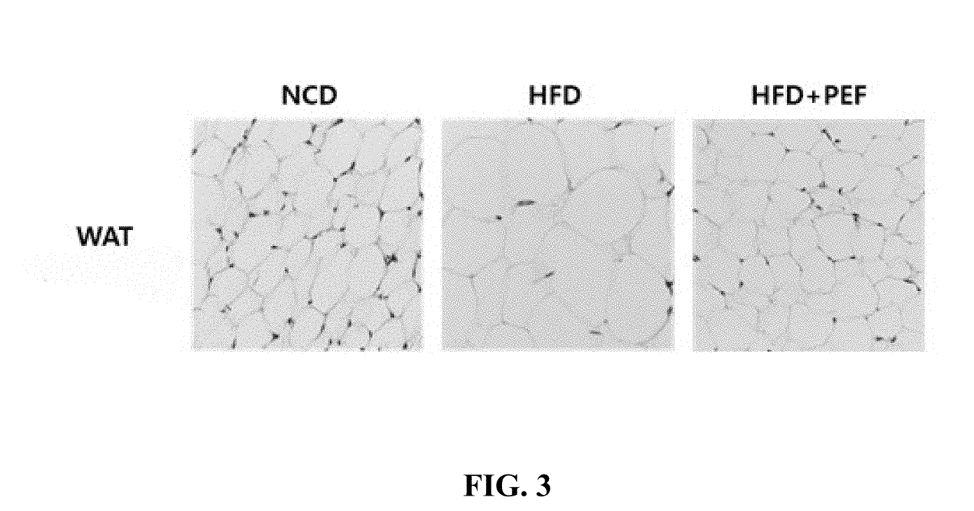

As the result of checking the effect of the water extract of Pleurotus eryngii var. ferulea (PF.) on the morphology adipose tissue (WAT) with the microscope as shown in FIG. 3, the group (HFD) having high fat diet has the adipose cell size significantly increased compared to the normal diet group (NCD), whereas the size of fat cells of the group which had high fat diet and the water extract of Pleurotus eryngii var. ferulea (PF. (HFD+PEF) was significantly decreased.

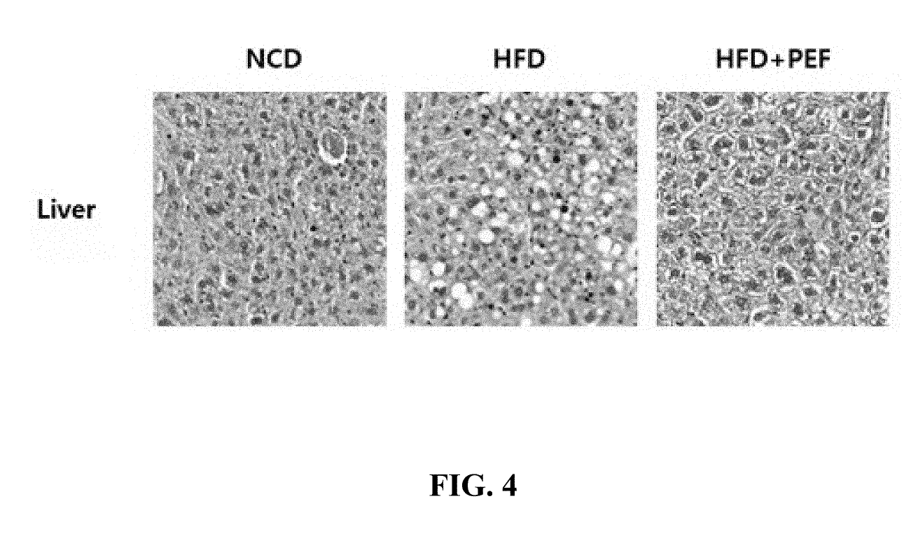

In addition, as the result of checking the inhibitory effect of water extract of Pleurotus eryngii var. ferulea (PF.) on the fat accumulation of the liver tissue with a microscope as shown in FIG. 4, the fat accumulation was distributed overall in the high fat diet group (HFD) compared to normal diet group (NCD), whereas the fat accumulation in the group which had high fat diet and the water extract of Pleurotus eryngii var. ferulea (PF.) (HFD+PEF) was significantly decreased near to the normal diet group.

Therefore, a water extract of Pleurotus eryngii var. ferulea (PF.) was found to have an effect of inhibiting fat accumulation caused by high fat diet.

### Example 2-4: Verification of Anti-Diabetic Effect

In order to check the anti-diabetic effect of a water extract of Pleurotus eryngii var. ferulea (PF.), the blood glucose content was measured by separating the plasma from the blood of the experimental animals. The blood of the experimental animals were extracted 8 weeks after the start of the experiment, the plasma was obtained by centrifuging the blood at 4° C. for 10 minutes, and the glucose in plasma was measured by using a glucose meter (Accu-chek performa nan, Accu-chek).

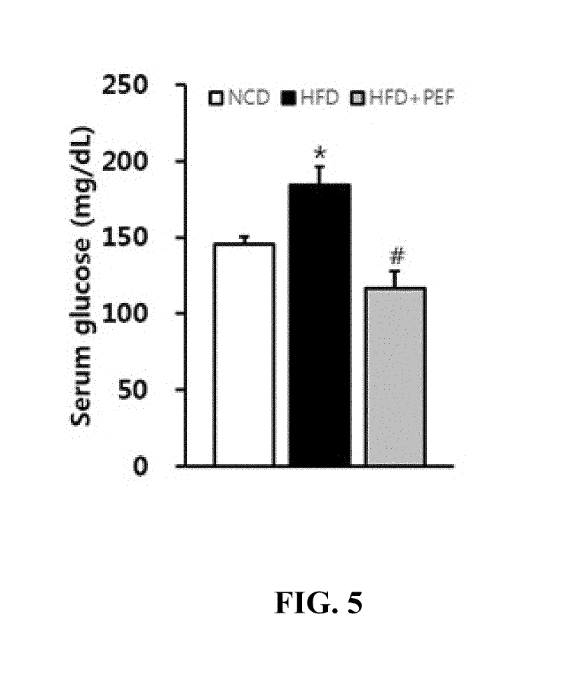

As the result of measuring glucose as shown in FIG. 5, the glucose of the high fat diet group (HFD+PEF) was increased compared to that of the normal diet group (NCD), whereas the glucose of the group which had high fat diet and the water extract of Pleurotus eryngii var. ferulea (PF.) (HFD+PEF) was decreased.

Therefore, a water extract of Pleurotus eryngii var. ferulea (PF.) was found to have an effect of inhibiting increase in glucose caused by high fat diet.

The examples of various formulations containing a water extract of Pleurotus eryngii var. ferulea (PF.) of the present invention as an active ingredient are described in the below, but the preparation of the present invention is not limited to the same.

### Preparation Example 1. Preparation of Powder

20 mg of water extract powder of Pleurotus eryngii var. ferulea (PF.)

100 mg of lactose

10 mg of talc

The above components were mixed and filled in an airtight bag to prepare a powder.

### Preparation Example 2. Preparation of Tablet

10 mg of water extract powder of Pleurotus eryngii var. ferulea (PF.)

100 mg of corn starch

100 mg of lactose

2 mg of magnesium stearate

The above components were mixed and prepared into a tablet according to the conventional method of manufacturing tablets to prepare tablets.

### Preparation Example 3. Preparation of Capsules

10 mg of water extract powder of Pleurotus eryngii var. ferulea (PF.)

3 mg of crystalline cellulose

14.8 mg of lactose

0.2 mg of magnesium stearate

The above components were mixed and filled in a gelatin capsule according to a conventional capsule production method to prepare a capsule.

### Preparation Example 4. Preparation of Liquid Preparation

20 mg of water extract powder of Pleurotus eryngii var. ferulea (PF.)

10 g of isomerized glucose

5 g of mannitol

Proper Quantity of Purified Water

According to the conventional solution preparation method, each ingredient was added to the purified water and dissolved, and a proper amount of lemon flavor was applied. The above ingredients were mixed and purified water was added to control the total volume to 100 mL, the mixture was filled into a brown bottle and sterilized to prepare a liquid preparation.

### Preparation Example 5. Preparation of an Injection

10 mg of water extract powder of Pleurotus eryngii var. ferulea (PF.)

180 mg of mannitol

3000 mg of injectable sterile distilled water

25 mg of Na₂HPO₄, 12H₂O

An injection is made at the above content for one ampoule (2 mL) according to the conventional method for preparing injections.

### Preparation Example 6. Preparation of Health Functional Food

1000 mg of water extract powder of Pleurotus eryngii var. ferulea (PF.)

Proper Quantity of Vitamin Mixture

70 μg of vitamin A acetate

1.0 mg of vitamin E

0.13 mg of vitamin B1

0.15 mg of vitamin B2

0.5 mg of vitamin B6

0.2 μg of vitamin B12

10 mg of vitamin C

10 μg of biotin

1.7 mg of nicotinamide

50 μg of folic Acid

0.5 mg of Calcium pantothenate

Suitable Amount of Mineral Mixture

1.75 mg of ferrous sulfate

0.82 mg of zinc oxide

25.3 mg of magnesium carbonate

15 mg of monopotassium phosphate

55 mg of potassium Phosphate dibasic

90 mg of potassium citrate

100 mg of calcium carbonate

24.8 mg of magnesium chloride

The composition ratio of the vitamin and mineral mixtures were set by mixing compositions relatively suitable for health food as a preferred embodiment, but the mixing ratio may be randomly changed for working, and after mixing said ingredients according to a conventional method for manufacturing health foods, a granule is prepared and can be used in preparation of a health food composition.

### INDUSTRIAL APPLICABILITY

The present invention as described above is useful invention in terms of medicine manufacture and health food industry by having an excellent effect of being provided as a pharmaceutical composition for preventing and treating or a health functional food for preventing and alleviating metabolic diseases such as obesity and diabetes by confirming that a water extract powder of Pleurotus eryngii var. ferulea (PF.) has an effect of inhibiting fat accumulation and glucose increase caused by intake of high fat diet.

1. A pharmaceutical composition for preventing and treating metabolic diseases such as obesity or diabetes, containing a water extract powder of Pleurotus eryngii var. ferulea (PF.) as an active ingredient.

2. The pharmaceutical composition of claim 1, wherein the pharmaceutical composition is characterized by being formulated into any one of powders, tablets, hard and soft capsules, liquids and injections.

3. A functional health food for preventing and alleviating metabolic diseases such as obesity or diabetes, containing a water extract powder of Pleurotus eryngii var. ferulea (PF.) as an active ingredient.

4. The functional health food of claim 3, wherein the health functional food is characterized by being selected as any one of foods, beverages, gum, tea and vitamin complexes.
